(19) United States
(12) Patent Application Publication
(10) Pub. No.: US 2018/0014868 A1
(43) Pub. Date: Jan. 18, 2018

(54) SYSTEMS AND METHODS TO BLOCK OR INHIBIT GAS PROGRESSION DURING SPRAY CRYOTHERAPY

(71) Applicant: CSA Medical, Inc., Lexington, MA (US)

(72) Inventors: John P. O'Connor, Andover, MA (US); Brian M. Hanley, Reading, MA (US)

(21) Appl. No.: 15/648,397

(22) Filed: Jul. 12, 2017

Related U.S. Application Data
(60) Provisional application No. 62/361,576, filed on Jul. 13, 2016.
(60) Provisional application No. 62/414,099, filed on Oct. 28, 2016.

(51) Int. Cl. A61B 18/02; A61B 1/018; A61B 18/00

(52) U.S. Cl. CPC A61B 18/02; A61B 1/018; A61B 2018/00577; A61B 2018/00541; A61B 2018/0022; A61B 2018/00023; A61B 2018/0212; A61B 2018/00482

(57) ABSTRACT

The present disclosure relates generally to the field of cryoablation. In particular, the present disclosure relates to cryoablation systems (e.g., cryospray systems, cryogenic ablation, cryosurgery systems etc.) that prevent or significantly inhibit cryospray gases from accumulating and progression distally beyond a specific region within a body lumen.

### PRIORITY

This application claims the benefit of priority under 35 U.S.C. §119 to U.S. Provisional Patent Application Ser. No. 62/361,576, filed Jul. 13, 2016 and U.S. Provisional Application Ser. No. 62/414,099, filed Oct. 28, 2016, both of which are incorporated by reference in their entirety and for all purposes.

### FIELD

The present disclosure relates generally to the field of cryoablation. In particular, the present disclosure relates to cryoablation systems (e.g., cryospray systems, cryogenic ablation, cryosurgery systems etc.) that prevent or significantly inhibit cryospray gases from accumulating and progressing distally beyond a specific region within a body lumen.

### BACKGROUND

Cryoablation is a surgical procedure in which diseased, damaged or otherwise undesirable tissue (collectively referred to herein as “target tissue”) is destroyed by focal delivery of a cryogen spray under pressure. These systems are typically referred to as cryoablation systems, cryospray systems, cryospray ablation systems, cryosurgery systems, cryosurgery spray systems and/or cryogen spray ablation systems. As typically used, “cryogen” refers to any fluid (e.g., gas, liquefied gas or other fluid known to one of ordinary skill in the art) with a sufficiently low boiling point (i.e., below approximately −153° C.) for therapeutically effective use during a cryogenic surgical procedure. Suitable cryogens may include, for example, liquid argon, liquid nitrogen and liquid helium. Pseudo-cryogens such as liquid carbon dioxide and liquid nitrous oxide that have a boiling temperature above −153° C. but still very low (e.g., −89° C. for liquid N₂O) may also be used.

During operation of a cryoablation system, a medical professional (e.g., clinician, technician, physician, surgeon etc.) directs a cryogen spray onto the surface of a treatment area via a cryogen delivery catheter. The medical professional may target the cryogen spray visually through a video-assisted device or scope, such as a bronchoscope, endoscope, colonoscope or ureteroscope. Cryogen spray exits the cryogen delivery catheter at a temperature ranging from 0° C. to −196° C., causing the target tissue to freeze or “cryofrost.” As liquid cryogens exit the cryogen delivery catheter and impact upon the target, they convert to a gaseous state with a significant increase in volume. For example, 1 cubic centimeter (cm³) of liquid nitrogen converts to 694 cm³ of nitrogen gas at body temperature. If not properly vented from the patient, these expanding gases cause undue distention and may have life-threatening consequences, including, for example, pneumothorax of the lungs and perforations of the upper or lower gastrointestinal (GI) tract.

There is an ongoing need for cryoablation systems and methods which block the distal progression of expanding cryospray gases within body lumens, and actively or passively vent such gases outside of the patient.

### SUMMARY

The present disclosure, in its various aspects, meets an ongoing need in the field of cryoablation for a system that prevents or significantly inhibits cryospray gases from accumulating and progressing distally beyond a specific region of a body lumen.

In one aspect, the present disclosure provides a cryoablation system, comprising an endoscope which includes a proximal portion, a distal portion, and at least one working channel extending therebetween; a cryogen delivery catheter disposed within a working channel of the endoscope, the cryogen delivery catheter comprising a proximal end, distal end configured for the output of cryogen spray, and a lumen extending therebetween; and an expandable member moveable between a deflated configuration and an inflated configuration and including a conduit comprising a proximal inlet and a distal outlet, wherein the distal outlet is fluidly connected to an interior of the expandable member. The expandable member may be configured to extend distally beyond the distal portion of the endoscope. The expandable member may be disposed within a working channel of the endoscope. The expandable member may move from the deflated configuration to the inflated configuration by flowing a fluid into the interior of the expandable member. The expandable member may move from the inflated configuration to the deflated configuration by flowing a fluid through the conduit and out of the interior of the expandable member. The expandable member may comprise a non-compliant or semi-compliant material. For example, the non-compliant or semi-compliant material may comprise a polymer including, but not limited to, PEBAX, PET, PEN, PBT, PEEK, Hytrel, polyurethane and nylon. In addition, or alternatively, the expandable member may comprises a compliant material including, but not limited to, silicone rubbers, polyurethanes, butyl rubbers, latexes, styrene-isobutylene-styrene block copolymers and EPDM. The expandable member may be a balloon. The expandable member may conform to the shape of a body lumen when in the inflated configuration. The expandable member may prevent or substantially inhibit gas progression distally beyond the expandable member. The endoscope may include a second working channel configured for active or passive venting of a gas therethrough. The cryoablation system may further include a vent tube configured for passive or active venting of a gas therethrough. The vent tube may be configured to be disposed within the working channel of the endoscope. The vent tube may extend distally beyond the distal portion of the endoscope. The vent tube may extend distally beyond the distal portion of the endoscope and through the expandable member. The cryoablation system may further include a vent tube configured for passive or active venting of a gas therethrough, wherein the vent tube is independent of the endoscope working channel.

In another aspect, the present disclosure provides a method, comprising introducing an endoscope into a lumen of a patient; positioning a distal portion of the endoscope at a first location within the lumen; introducing an expandable member into the lumen; moving the expandable member from a deflated configuration to an inflated configuration; retracting the endoscope to position the distal portion of the endoscope at a second location within the lumen, wherein the second location is proximal to the first location; introducing a cryogen delivery catheter into the lumen such that a distal end of the cryogen delivery catheter extends distally beyond the distal portion of the endoscope; delivering a cryogen spray through the cryogen delivery catheter to a target tissue at the second location; and venting the lumen. The expandable member may be introduced through a working channel of the endoscope into the lumen. There may be more than one expandable member. The expandable member(s), may be inflated and deflated together or in unison, and may share inflation and/or deflation lumen, or may have dedicated inflation and/or deflation lumen. The cryogen delivery catheter may be introduced through a working channel of the endoscope. The lumen may be actively and/or passively vented. The lumen may be vented proximal and distal to the expandable member. The body lumen may include, but is not limited to, the lower gastrointestinal system, the upper gastrointestinal system and the respiratory system.

In yet another aspect, the present disclosure provides a method comprising blocking a body lumen at a location distal to a target tissue; delivering a cryogen spray to the target tissue; and venting a gas produced by the cryogen spray. One or more of the foregoing steps and features may be applicable.

In another aspect, the present disclosure provides a cryoablation system that includes a cryogen delivery catheter comprising a proximal end, a distal end including an outlet for cryogen, and a lumen extending therebetween; a single-lumen conduit disposed within the lumen, the single-lumen conduit comprising a distal portion extending distally beyond the cryogen outlet at the distal end of the cryogen delivery catheter, wherein the distal portion includes at least one port in fluid communication with the single-lumen conduit; and an expandable member disposed about the distal portion of the single-lumen conduit and defining an interior, wherein the expandable member is moveable between an unexpanded (e.g., deflated) configuration and an expanded (e.g., inflated) configuration. The expandable member may move from the unexpanded configuration to the expanded configuration by flowing a fluid through the single-lumen conduit, and the at least one port, into the interior of the expandable member. Similarly, the expandable member may move from the expanded configuration to the unexpanded configuration by flowing a fluid from the interior of the expandable member through the at least one port and the single-lumen conduit. The cryoablation system may further include a vent tube disposed within the lumen of the cryogen delivery catheter. A distal portion of the vent tube may pass through and extend distally beyond the expandable member. The expandable member may include a balloon comprising a non-compliant or semi-compliant material, including, but not limited to, PEBAX, PET, PEN, PBT, PEEK, Hytrel, polyurethane and nylon.

In yet another aspect, the present disclosure provides a cryoablation system that includes a cryogen delivery catheter comprising a proximal end, a distal end configured for the output of cryogen, and a lumen extending therebetween; a multi-lumen conduit disposed within the lumen of the cryogen delivery catheter, the multi-lumen conduit comprising a distal portion extending distally beyond the distal end of the cryogen delivery catheter, wherein the distal portion includes at least one first lumen port in fluid communication with a first lumen of the multi-lumen conduit and at least one second lumen port in fluid communication with a second lumen of the multi-lumen conduit; and an expandable member disposed about the distal portion of the multi-lumen conduit, wherein the expandable member is moveable between a deflated (e.g., unexpanded) configuration and an inflated (e.g., expanded) configuration. The expandable member may move from the deflated configuration to the inflated configuration by flowing a fluid through the first lumen of the multi-lumen conduit, and the at least one first lumen port, into the interior of the expandable member. The expandable member may move from the inflated configuration to the deflated configuration by flowing a fluid from the interior of the expandable member through the at least one second lumen port and second lumen of the multi-lumen conduit. The cryoablation system may further include a vent tube. The vent tube may be disposed within the lumen of the cryogen delivery catheter. A distal portion of the vent tube may pass through and extend distally beyond the expandable member. The expandable member may include a balloon comprising a non-compliant or semi-compliant material, including, but not limited to, PEBAX, PET, PEN, PBT, PEEK, Hytrel, polyurethane and nylon.

In another aspect of the present disclosure, a cryoablation system may include a cryogen delivery catheter having a proximal end, a distal end configured for the output of cryogen, and a lumen extending therebetween. A multi-lumen conduit may be disposed within the lumen of the cryogen delivery catheter. The multi-lumen conduit may include a distal portion extending distally beyond the distal end of the cryogen delivery catheter. The distal portion may include at least one port in fluid communication with a first lumen of the multi-lumen conduit and at least one port in fluid communication with a second lumen of the multi-lumen conduit. A first expandable member may be disposed about the distal portion of the multi-lumen conduit. The first expandable member may define an interior. The first expandable member may be moveable between a deflated configuration and an inflated configuration.

In another aspect, the first expandable member may be moveable from the deflated configuration to the inflated configuration by flowing a fluid through the first lumen of the multi-lumen conduit, and the at least one first lumen port, into the interior of the expandable member. The first expandable member may be moveable from the inflated configuration to the deflated configuration by flowing a fluid from the interior of the expandable member through the at least one second lumen port and second lumen of the multi-lumen conduit. A vent tube may be disposed within the lumen of the cryogen delivery catheter, wherein a distal portion of the vent tube passes through and extends distally beyond the first expandable member. The first expandable member may include a non-compliant or semi-compliant material. The non-compliant or semi-compliant material may include a polymer selected from the group consisting of PEBAX, PET, PEN, PBT, PEEK, Hytrel, polyurethane and nylon.

In another aspect, a second expandable member may be disposed about the distal portion of the multi-lumen conduit and define an interior, wherein the second expandable member is moveable between a deflated configuration and an inflated configuration. The multi-lumen conduit may include the first lumen in fluid communication with interior of the first expandable member. The multi-lumen conduit may include the second lumen in fluid communication with the interior of the second expandable member. The multi-lumen conduit may include a third lumen in fluid communication with the interior of the first expandable member and the second expandable member. The first expandable member may transition from the deflated configuration to the inflated configuration by flowing a fluid through the first lumen to the interior of the first expandable member. The second expandable member may transition from the deflated configuration to the inflated configuration by flowing a fluid through the second lumen to the interior of the second expandable member. The first and second expandable members may transition from the inflated configuration to deflated configuration by flowing a fluid from interior of the first and second expandable members through the third lumen.

In still another aspect, the present disclosure provides a cryoablation system that includes a cryogen delivery catheter comprising a proximal end, a distal end configured for the output of cryogen, and a lumen extending therebetween; a multi-lumen conduit (e.g., having a plurality of lumens) disposed within the lumen of the cryogen delivery catheter; and first and second expandable members disposed about the distal portion of the multi-lumen conduit, wherein the first and second expandable members are moveable between a deflated configuration and an inflated configuration. The first expandable member may move from the deflated configuration to the inflated configuration by flowing a fluid through the first lumen of the multi-lumen conduit into the interior of the first expandable member. The second expandable member may move from the deflated configuration to the inflated configuration by flowing a fluid through the second lumen of the multi-lumen conduit into the interior of the second expandable member. The first and second expandable members may move from the inflated configuration to deflated configuration by flowing a fluid from the interiors of the first and second expandable members through a third lumen of the multi-lumen conduit. The cryoablation system may also include a vent tube disposed within the lumen of the cryogen delivery catheter, wherein a distal portion of the vent tube passes through and extends distally beyond the first expandable member. In addition, or alternatively, the cryoablation system may include a vent tube disposed within the lumen of the cryogen delivery catheter, wherein a distal portion of the vent tube passes through and extends distally beyond the second expandable member. The first and second expandable members may include a balloon comprising a non-compliant or semi-compliant material, including, but not limited to, PEBAX, PET, PEN, PBT, PEEK, Hytrel, polyurethane and nylon. The multi-lumen conduit may include a distal portion extending distally beyond the distal end of the cryogen delivery catheter, wherein the distal portion includes at least one first lumen port in fluid communication with a first lumen of the multi-lumen conduit; at least one second lumen port in fluid communication with a second lumen of the multi-lumen conduit; and at least two third lumen ports in fluid communication with a third lumen of the multi-lumen conduit.

In another aspect, a cryoablation system may include a cryogen delivery catheter having a proximal end, a distal end including an outlet for cryogen, and a lumen extending therebetween. The system may include a conduit having at least a first lumen disposed within the lumen of the cryogen delivery catheter. The conduit may have a distal portion extending distally beyond the cryogen outlet at the distal end of the cryogen delivery catheter. The distal portion may include at least one first lumen port in fluid communication with the first lumen. A first expandable member may be disposed about the distal portion of the conduit and define an interior. The first expandable member may be moveable between an unexpanded configuration and an expanded configuration. The first expandable member may move from the unexpanded configuration to the expanded configuration by flowing a fluid through the first lumen, and the at least one first lumen port, into the interior of the first expandable member. The first expandable member may move from the expanded configuration to the unexpanded configuration by flowing a fluid from the interior of the first expandable member through the at least one first lumen port and the first lumen. A vent tube may be disposed within the lumen of the cryogen delivery catheter. A distal portion of the vent tube may pass through and extend distally beyond the first expandable member. The first expandable member may be a balloon and may be a non-compliant or semi-compliant material. The non-compliant or semi-compliant material may be a polymer selected from the group consisting of PEBAX, PET, PEN, PBT, PEEK, Hytrel, polyurethane and nylon. The outlet for cryogen may include at least one aperture disposed around a circumference of the cryogen delivery catheter. The conduit may include a second lumen disposed within the lumen of the cryogen delivery catheter. The distal portion of the conduit may include at least one second lumen port in fluid communication with the second lumen. The first expandable member may move from the expanded configuration to the unexpanded configuration by flowing a fluid from the interior of the first expandable member through the at least one second lumen port and the second lumen. The first expandable member may conform to the shape of a body lumen when in the inflated configuration and may prevent or substantially inhibit gas progression distally beyond the expandable member.

In another aspect, a second expandable member may be disposed about the distal portion of the conduit and may define an interior. The second expandable member may be moveable between an unexpanded configuration and an expanded configuration. The conduit may include a third lumen in fluid communication with the interior of the first expandable member and the second expandable member. The second lumen may be in fluid communication with the interior of the second expandable member. The first lumen may be in fluid communication with the interior of the first expandable member. The first expandable member may transition from the unexpanded configuration to the expanded configuration by flowing a fluid through the first lumen to the interior of the first expandable member. The second expandable member may transition from the unexpanded configuration to the expanded configuration by flowing a fluid through the second lumen to the interior of the second expandable member. The first expandable member and the second expandable member may transition from the expanded configuration to the unexpanded configuration by flowing a fluid from the interior of the first and second expandable members through the third lumen. A vent tube may be disposed within the lumen of the cryogen delivery catheter. A distal portion of the vent tube may pass through and extend distally beyond the first expandable member and distally beyond the second expandable member. The vent tube may be independent of the working channel of the endoscope.

### BRIEF DESCRIPTION OF THE DRAWINGS

Non-limiting embodiments of the present disclosure are described by way of example with reference to the accompanying figures, which are schematic and not intended to be drawn to scale. In the figures, each identical or nearly identical component illustrated is typically represented by a single numeral. For purposes of clarity, not every component is labeled in every figure, nor is every component of each embodiment shown where illustration is not necessary to allow those of ordinary skill in the art to understand the disclosure. In the figures:

FIGS. 1A-1C provide perspective views of cryoablation systems deployed within the lower gastrointestinal tract, according to one embodiment of the present disclosure.

FIGS. 2A-2C provide perspective views of cryoablation systems deployed within the upper gastrointestinal tract, according to another embodiment of the present disclosure.

FIGS. 3A-3B provide perspective views of cryoablation systems deployed within the respiratory tract, according to yet another embodiment of the present disclosure.

FIG. 4 provides a perspective view of a balloon cryoablation system, according to an embodiment of the present disclosure.

FIGS. 5A-5B provide perspective (FIG. 5A) and cross-section (FIG. 5B) views of a balloon cryoablation system, according to another embodiment of the present disclosure.

FIGS. 6A-6C provide perspective (FIG. 6A) and cross-section (FIGS. 6B and 6C) views of a dual-balloon cryoablation system, according to another embodiment of the present disclosure.

FIG. 7 provides a perspective view of another embodiment of the balloon cryoablation system of FIG. 4.

### DETAILED DESCRIPTION

The present disclosure is not limited to the particular embodiments described. The terminology used herein is for the purpose of describing particular embodiments only, and is not intended to be limiting beyond the scope of the appended claims. Unless otherwise defined, all technical terms used herein have the same meaning as commonly understood by one of ordinary skill in the art to which the disclosure belongs.

Although embodiments of the present disclosure are described with specific reference to cryoablation systems for use within the upper and lower GI tracts and respiratory system, it should be appreciated that such systems and methods may be used in a variety of other body passageways, organs and/or cavities, such as the vascular system, urogenital system, lymphatic system, neurological system and the like. It should also be appreciated that the systems of the present disclosure are not necessarily limited to cryoablation procedures, but may be employed in other medical procedures in which it is desirable to employ an expandable member to block the progress of a substance or medical instrument further into a body passage.

As used herein, the singular forms “a,” “an,” and “the” are intended to include the plural forms as well, unless the context clearly indicates otherwise. It will be further understood that the terms “comprises” and/or “comprising,” or “includes” and/or “including” when used herein, specify the presence of stated features, regions, steps elements and/or components, but do not preclude the presence or addition of one or more other features, regions, integers, steps, operations, elements, components and/or groups thereof.

As used herein, the term “distal” refers to the end farthest away from the medical professional when introducing a device into a patient, while the term “proximal” refers to the end closest to the medical professional when introducing a device into a patient.

As used herein, the term “expandable” refers to the ability to increase in diameter from a “collapsed” or “deflated” configuration to an “expanded” or “inflated” configuration. As used herein, “diameter” refers to the distance of a straight line extending between two points and does not necessarily indicate a particular shape.

As used herein, the term “passive venting” refers to the unassisted egress of gases from within a body lumen to an external location, through body lumen and natural orifice or through a ventilation tube passing through the same. As used herein, the term “active venting” refers to the mechanically-assisted egress (e.g., via a suction source) of gases from with a body lumen to an external location through a ventilation tube.

As used herein, the term “conduit” may refer to a member containing one or more lumens (e.g., inflation, deflation, and/or venting lumens). Alternatively, a conduit may refer to multiple members containing one or more lumens (e.g., multiple members alongside each other each containing inflation, deflation, and/or venting lumens).

The present disclosure generally provides cryoablation systems configured to block the distal progression of materials and/or substances, including, but not limited to, cryospray gases (hereafter referred to as “cryospray”) within a body lumen, and simultaneously vent such cryospray to prevent their accumulation. Exemplary cryoablation systems in which the present disclosure may be implemented include, but are not limited to, those systems described in U.S. Pat. Nos. 9,301,796, and 9,144,449, and U.S. patent application Ser. Nos. 11/956,890, 12/022,013, 14/012,320, and 14/869,814, each of which are herein incorporated by reference in their entirety.

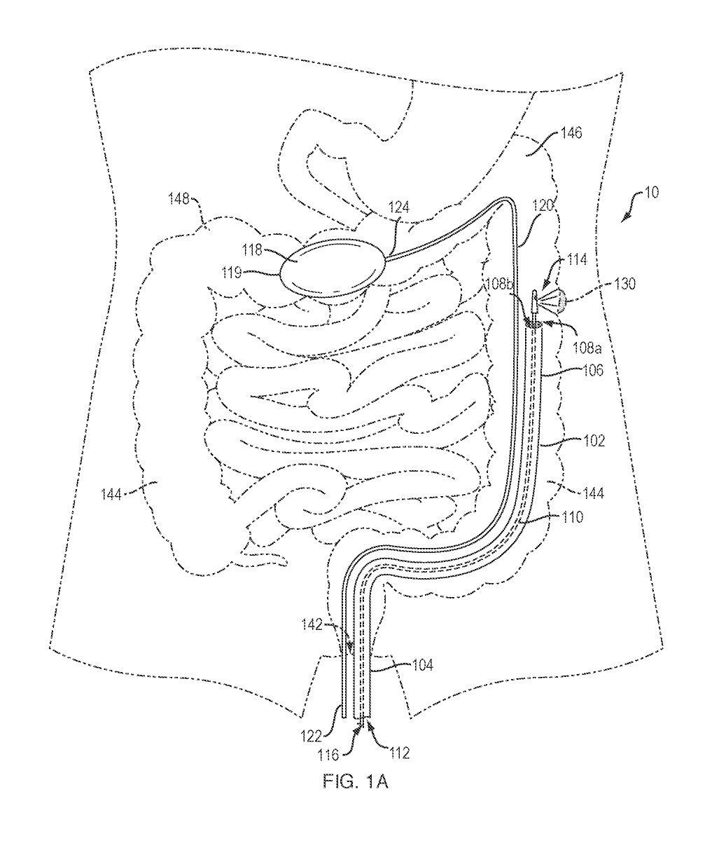

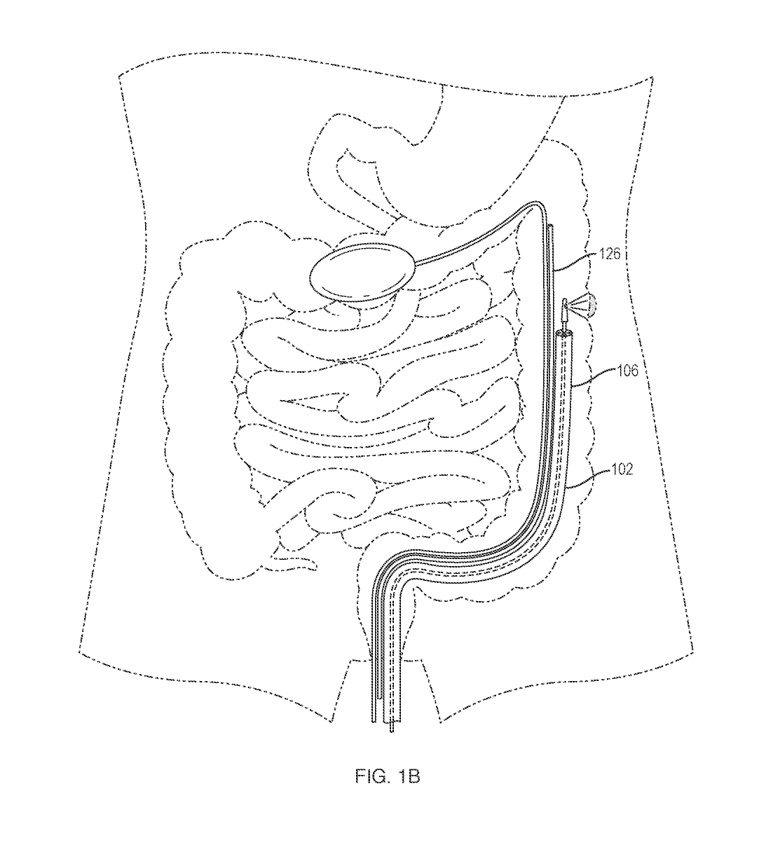

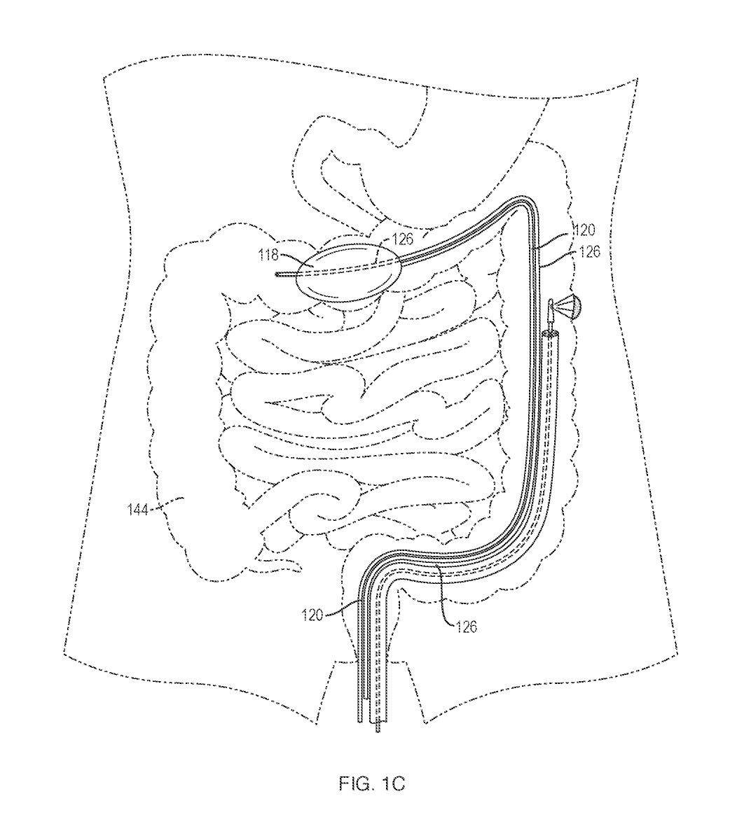

Referring to FIG. 1A, in one embodiment a cryoablation system 10 of the present disclosure may include an endoscope 102 comprising a proximal portion 104, a distal portion 106 and a first working channel 108a extending therebetween. The endoscope 102 may include any appropriate size, although smaller diagnostic endoscopes are preferably used to facilitate navigation within body passageways and facilitate patient comfort. The endoscope 102 may further include a second working channel 108b configured to vent the cryo spray delivered from the cryogen delivery catheter 110 (discussed below). In one embodiment, the second working channel 108b is configured for passive venting of the cryospray. In another embodiment, the second working channel 108b is connected to a suction source (e.g., pump, not depicted) to facilitate active venting of the cryospray. In addition, or alternatively, a vent tube may be passed through the second working channel 108b for active or passive venting of the cryospray. As will be understood by those in the art, the diameter of the second working channel 108b through which the cryospray passively or actively vents must be adequate to ensure that organ or body cavity distention does not occur. In addition, or alternatively, a tube (e.g., sleeve) may be disposed around or independent of an outer surface of the endoscope for passive or active venting of cryospray, leaving the working channel(s) of the endoscope available for other medical tools. Passive venting of cryospray may also be achieved in the absence of an active or passive tube or working channel by managing the body lumen to maintain proper circulation and egress of gases. For example, the respective entry point (e.g., esophagus, rectum etc.) of the body lumen may be maintained in an open configuration to ensure that internal air pressure at or near the site of the cryoablation procedure remains equal to the atmospheric pressure (e.g., the pressure outside the body). In addition, or alternatively, the position of the patient on the operating table may be adjusted (i.e., lying flat, prone, inclined, declined, on their left or right side) to prevent the lumen from partially or completely collapsing under the patient's own weight.

A cryogen delivery catheter 110 may be disposed within the first working channel 108a of the endoscope 102. The cryogen delivery catheter 110 may include a proximal end 112, a distal end 114 and a lumen 116 extending therebetween. The distal end 114 may include closed or open-ended configurations, with or without side apertures disposed around a portion or whole of the circumference thereof. Cryogen (e.g., liquid nitrogen) may be delivered from an external storage tank (not depicted) connected to the proximal end 112 of the cryogen delivery catheter 110, through the lumen 116 to exit through side and/or end aperture(s) at distal end 114. The distal end 114 of the cryogen delivery catheter 110 may include one or more apertures configured to convert the cryogen flowing through the lumen 116 into a pressurized, e.g., low pressure cryogen spray. The cryogen delivery catheter may include various sensors, e.g., temperature sensor, and may be connected to a console with controls that may be necessary or useful to control and monitor a cryospray procedure, including for example regulation of cryogen flow based on temperature feedback, other procedural parameters, venting, etc. In one embodiment, the cryogen delivery catheter may be constructed of three layers of flexible polyimide, surrounded by a stainless steel braid, which is coated with an outer layer of Pebax. As understood by those in the art, extrusion of Pebax over the stainless steel braid allows the Pebax to wick through the pitch of the steel braid, helping to prevent kinking, breaking or delaminating during retroflex of the cryogen delivery catheter.

As apparent to those of skill in the art, the cryogen delivery catheter of the present disclosure may include a variety of suitable materials and/or dimensions depending on the demands of the particular application. As used herein, the term “retroflex” refers to the ability of a medical instrument to bend or turn approximately 180° about a radius of curvature or 1 inch or less.

The cryoablation system 10 may further include an expandable member 118 configured to move from a deflated (i.e., collapsed) configuration to an inflated (i.e., expanded) configuration by flowing an inflation fluid into the interior of the expandable member through a conduit 120. The conduit 120 may include a proximal inlet 122 fluidly connected to an external fluid source (not shown) and a distal outlet 124 fluidly connected to the interior of the expandable member 118. In one embodiment, the conduit 120 may include a dual-lumen configuration for separate inflow (e.g., inflation lumen) and outflow (e.g., deflation lumen) of inflation fluid. In another embodiment, the conduit may include a single lumen for inflow and outflow of inflation fluid, and a separate/alternate working channel or guidewire lumen. In one embodiment, expandable member 118 may move from a deflated configuration to an expanded configuration by flowing the inflation fluid into the interior of the expandable member 118 through the conduit 120. The expandable member 118 may return to the deflated configuration by flowing the inflation fluid from the interior of the expandable member 118 back to the external fluid source through the conduit 120. The inflation fluid may include a variety of physiologically inert liquids (e.g., buffered solutions such as sterile saline) or gases (e.g., air, oxygen, nitrogen, hydrogen, carbon dioxide, helium etc.) as are known in the art. It should be appreciated that the inflatable member should be positioned sufficiently distal to the cryogen delivery catheter such that the cryospray does not cause the liquids within the expandable member to freeze, or the gases within the expandable member to condense to the point that the expandable member contracts/deflates. Alternatively, liquids resistant to freezing at cryogen temperatures may be chosen so the expandable member can be located closer to the cryogen delivery catheter. In one embodiment, an inner or outer surface of the expandable member may include one or more temperature sensors and/or pressure gauges to allow the temperature of the expandable member to be monitored during the cryospray procedure.

It will be appreciated that the expandable member 118 may be provided in a variety of different inflated dimensions in order to block a range of lumen sizes. In one embodiment, the expandable member (e.g., balloon) may include a combination of elastomeric and semi-compliant to non-compliant materials, such as thermoplastics and/or thermosets. The semi-compliant nature of these materials is desirable in some embodiments to ensure that the expandable member does not over-expand within the target body lumen. Examples of thermoplastics include polyolefins; polyamides (e.g., nylon, such as nylon 12, nylon 11, nylon 6/12, nylon 6, nylon 66); polyesters (e.g., polyethylene terephthalate (PET), polybutylene terephthalate (PBT), polyethylene naphthalate (PEN), polytrimethylene terephthalate (PTT)); polyethers; polyurethanes; polyvinyls; polyacrylics; fluoropolymers; copolymers and block copolymers thereof, such as block copolymers of polyether and polyamide (e.g., PEBAX®); and mixtures thereof. Examples of thermosets include elastomers (e.g., EPDM), epichlorohydrin, polyureas, nitrile butadiene elastomers and silicones. Other examples of thermosets include epoxies and isocyanates. Biocompatible thermosets may also be used. Biocompatible thermosets include, for example, biodegradable polycaprolactone, poly(dimethylsiloxane) containing polyurethanes and ureas and polysiloxanes. Ultraviolet curable polymers, such as polyimides and acrylic or methacrylic polymers and copolymers can also be used. Other examples of polymers that can be used in balloons include polyethylenes, polyethylene ionomers, polyethylene copolymers, polyetheretherketone (PEEK), thermoplastic polyester elastomers (e.g., Hytrel®) and combinations thereof. Other polymers are described, for example, in U.S. Pat. Pub. No. 2005/0043679, filed on Aug. 21, 2003, entitled “Medical Balloons,” the disclosure of which is incorporated in its entirety herein by reference. Expandable members may be folded, pleated and/or covered by a sheath until deployed to protect the expandable member and facilitate delivery within/through body lumens. Radiopaque materials may be incorporated into or onto the compliant, semi-compliant or non-compliant materials to allow the location of the expandable member to be visualized with systems capable of detection of radiopaque materials within the patient.

In another embodiment, a compliant expandable member may be desirable to establish and maintain firm contact with the tissue wall of amorphous and/or asymmetrically shaped lumens. As compared to non-compliant or semi-compliant materials, an expandable member formed from a compliant material will expand indefinitely (i.e., does not have a fixed final diameter). These expandable members are composed of materials with compliances preferably in the range of 10% to 800%, and more preferably in the range of 50% to 200%. Examples of compliant materials include elastomers such as silicone rubber, ethylene-propylene-diene copolymers, butyl rubber, styrene-isobutylene-styrene copolymers, urethanes, and latexes, among others.

In another embodiment, the cryogen delivery catheter may be used independent of an endoscope. For example, the cryogen delivery catheter 110 may include a steerable distal end 114 with a camera and light source to allow the medical professional to navigate through the body lumen to the target tissue site. The cryogen delivery catheter may include a working channel, separate from the lumen 116, through which an expandable member 118 may be introduced and deployed to a site distally beyond the target tissue. Alternatively, the expandable member 118 may be introduced and deployed into the body lumen through a separate delivery tube or sheath, independent of the cryogen delivery catheter. As discussed below, cryospray that advances distally beyond the expandable member 118 may be actively and/or passively vented through a vent tube 126 which passes through, and extends distally beyond, the expandable member 118. Cryospray proximal of the expandable member 118 may be vented through the working channel of the cryogen delivery catheter 110 and/or through a separate vent tube. In addition, or alternatively, cryospray proximal of the expandable member 118 may be passively vented without the assistance of a vent tube or working channel by managing the lumen and/or patient to maintain proper circulation and egress of gases, as discussed above.

In another embodiment, the cryoablation system may include a separate cryogen delivery catheter and endoscope (i.e., the cryogen delivery catheter is not disposed within a working channel of the endoscope). The expandable member 118 may be introduced through: 1) a working channel of the endoscope, 2) a working channel of the cryogen delivery catheter or 3) through a separate vent tube independent of both the cryogen delivery catheter and endoscope. As discussed below, cryospray that advances distally beyond the expandable member 118 may be actively and/or passively vented through a vent tube 126 which passes through, and extends distally beyond, the expandable member 118. Cryospray proximal of the expandable member may be vented through a working channel of the endoscope and/or through a separate vent tube. In addition, or alternatively, cryospray proximal of the expandable member 118 may be passively vented without the assistance of a vent tube or working channel by managing the lumen and/or patient to maintain proper circulation and egress of gases, as discussed above.

In use, and by way of example, the expandable member 118 may be introduced in the deflated configuration through the rectum 142 into the colon 144 distally beyond a target tissue 130. Once properly positioned (e.g., between the splenic flexure 146 and hepatic flexure 148), the expandable member 118 is moved to the inflated configuration such that at least a portion of the outer surface 119 contacts all, or substantially all, of the tissues about a circumference of the colon wall. With the expandable member positioned distally beyond the target tissue, the endoscope 102 is advanced into the colon 144 such that distal portion 106 is positioned adjacent to, or in the vicinity of, the target tissue 130. The cryogen delivery catheter 110 is then advanced distally beyond the distal portion 106 of the endoscope 102 such that the distal end 114 of the cryogen delivery catheter is adjacent to the target tissue 130.

Alternatively, the endoscope 102 may be advanced distally beyond the target tissue 130 such that the distal portion 106 of the endoscope is positioned at the desired expandable member deployment site within the colon 144. The expandable member 118 may then be advanced in a deflated configuration through a working channel (e.g., second working channel 108b) of the endoscope 102 and into the colon 144. Once properly positioned within the colon 144, the expandable member 118 is moved to the inflated configuration (as discussed above), and the endoscope 102 is retracted proximally to position the distal portion 106 adjacent to, or in the vicinity of, the target tissue 130. The cryogen delivery catheter 110 is then advanced distally beyond the distal portion 106 of the endoscope 102 through the first working channel 108a such that distal end 114 is adjacent to the target tissue, as discussed above.

The medical professional then releases cryogen from an external cryogen source (not depicted) through the lumen 116 of the cryogen delivery catheter. The cryogen warms and boils as it exits the cryogen delivery catheter, resulting in a cold cryospray emerging from the distal end 114 onto the target tissue 130. Freezing of the target tissue may be visualized by the acquisition of a white color, referred to as cryofrost. The white color indicates the onset of mucosal tissue freezing to initiate destruction of the target tissue. The medical professional may increase or decrease the duration of the cryospray treatment depending on the size and/or depth of the target tissue.

Cryospray that converts to gaseous form and accumulates within the lumen of the colon 144 may passively vent through the first working channel 108a of the endoscope to a location external to the patient. Alternatively, or in addition, cryospray that converts to gaseous form may be actively vented through the first working channel 108a under suction. As discussed above, passive venting of cryospray that converts to gaseous form may also be achieved in the absence of (or in addition to) active or passive venting through the first working channel 108a of the endoscope by managing the body lumen to maintain proper circulation and egress of gases. Once the cryoablation procedure is completed, and adequate venting is allowed to proceed, the expandable member is returned to the deflated configuration and the conduit with the expandable member, endoscope and cryogen delivery catheter are withdrawn from the patient.

FIG. 1B depicts an alternative embodiment, in which the cryoablation system depicted in FIG. 1A includes a vent tube 126 (e.g., cryogen decompression tube) to further assist in evacuation of the cryo spray, and other undesirable fluids and particles etc. Although the vent tube 126 is depicted as extending distally beyond the distal portion 106 of the endoscope 102, it will be appreciated that vent tube 126 may be positioned in a variety of locations relative to the distal portion 106 of the endoscope 102. The vent tube may connect via supplied accessory connection tubing (not depicted) to an external suction canister (not depicted) for active venting of the treatment area. Alternatively, the vent tube may include dual-lumens that provide both active (i.e., connected to a suction pump) and passive (direct to ambient atmosphere) vent paths.

FIG. 1C depicts another alternative embodiment, in which the vent tube 126 passes through, and extends distally beyond, the expandable member 118. Although the conduit 120 and vent tube 126 are depicted in a side-by-side configuration, in one embodiment, the vent tube may be disposed within and extend through the length of the conduit 120 and expandable member 118. The expandable member forms a tight seal around the outer surface of the vent tube 126 such that the respective lumens of the expandable member and vent tube remain separated and uncompromised. In this configuration, the vent tube may actively and/or passively vent cryospray that has advanced distally beyond the expandable member 118.

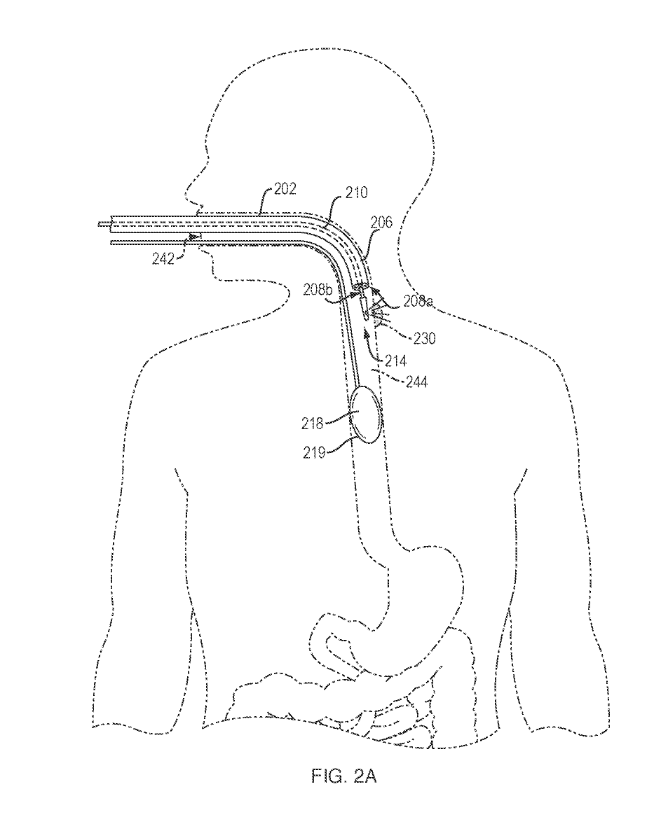

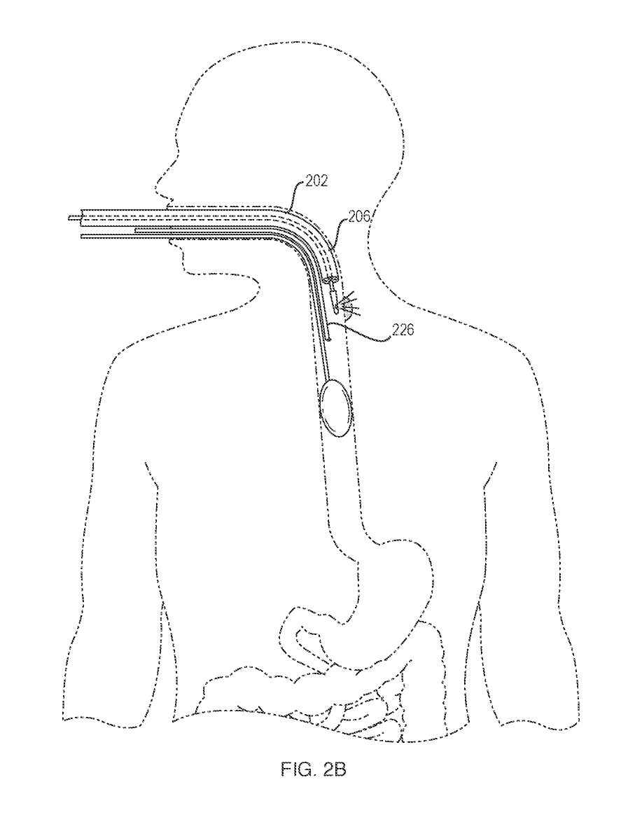

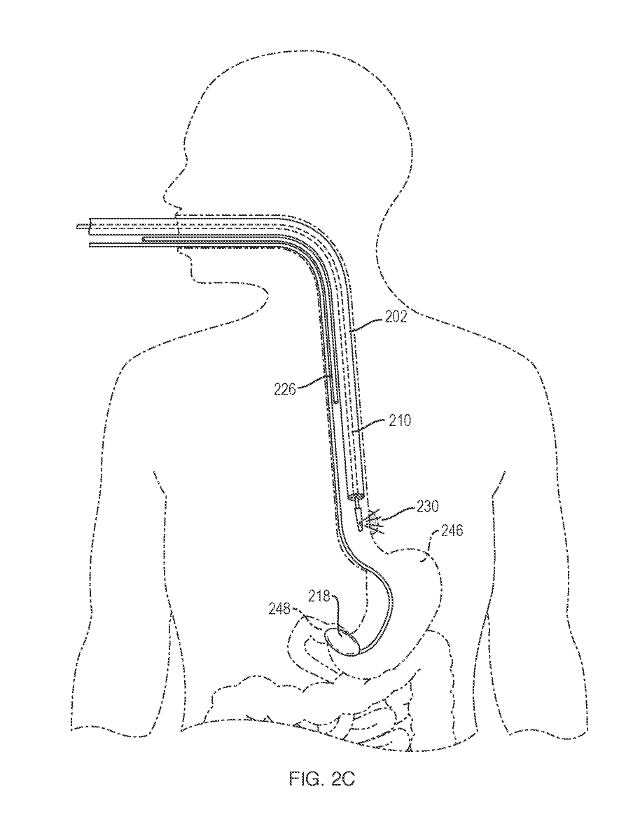

Referring to FIG. 2A, the expandable member 218 may be introduced in the deflated configuration through the mouth 242 into the esophagus 244 and distally beyond a target tissue 230. Once properly positioned within the esophagus 244, the expandable member 218 is moved to the inflated configuration such that at least a portion of the outer surface 219 contacts all, or substantially all, of the tissues about a circumference of the esophageal wall. With the expandable member positioned distally beyond the target tissue, the endoscope 202 is positioned within a portion of the esophagus 244 adjacent to, or in the vicinity of, the target tissue 230. The cryogen delivery catheter 210 is then advanced distally beyond the distal portion 206 of the endoscope 202 such that the distal end 214 is adjacent to the target tissue 230. Cryospray is then delivered to the target tissue as discussed above.

Alternatively, the endoscope 202 may be advanced distally beyond the target tissue 230 such that the distal portion 206 of the endoscope is positioned at the desired expandable member deployment site within the esophagus 244. The expandable member 218 may then be advanced in a deflated configuration through a working channel (e.g., second working channel 208b) of the endoscope 202 and into the esophagus 244. Once properly positioned within the esophagus 244, the expandable member 218 is moved to the inflated configuration (as discussed above), and the endoscope 202 is retracted proximally to position the distal portion 206 adjacent to, or in the vicinity of, the target tissue 230. The cryogen delivery catheter 210 is then advanced distally beyond the distal portion 206 of the endoscope 202 through the first working channel 208a such that distal end 214 is adjacent to the target tissue, as discussed above.

FIG. 2B depicts an alternative embodiment, in which the cryoablation system depicted in FIG. 2A includes a vent tube 226 to further assist in evacuation of the cryospray, and other undesirable fluids and materials. Although the vent tube 226 is depicted as extending distally beyond the distal portion 206 of the endoscope 202, it will be appreciated that vent tube may be positioned in a variety of locations within the esophagus to facilitate active and/or passive venting of cryospray as discussed above.

FIG. 2C depicts another alternative embodiment, in which the expandable member 218 is positioned within a distal region of the stomach 246 near the pylorus 248 to prevent or significantly inhibit cryospray from entering the duodenum. This placement of the expandable member 218 may allow the endoscope 202, cryogen delivery catheter 210 and vent tube 226 to access target tissues 230 at, or beyond, the gastroesophageal junction (GEJ). In alternative embodiments (not depicted), the vent tube may pass through, and extend distally beyond, the expandable member similar to FIG. 1C.

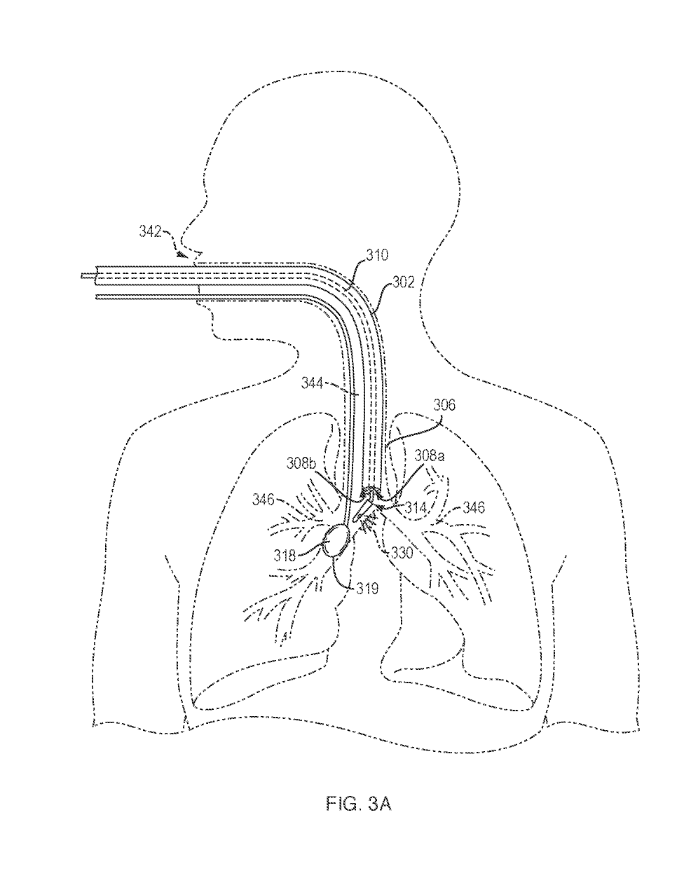

Referring to FIG. 3A, the expandable member 318 may be introduced in the deflated configuration through the mouth 342 into the trachea 344 and distally beyond a target tissue 330. Once properly positioned within a bronchial tube 346, the expandable member 318 is moved to the inflated configuration such that at least a portion of the outer surface 319 contacts all, or substantially all, of the tissues about a circumference of the bronchial tube. With the expandable member positioned distally beyond the target tissue 330, the endoscope 302 is positioned within a portion of the trachea 344 or bronchial tube 346 adjacent to, or in the vicinity of, the target tissue 330. The cryogen delivery catheter 310 is then advanced distally beyond the distal portion 306 of the endoscope 302 such that the distal end 314 is adjacent to the target tissue 330. Cryospray is then delivered to the target tissue as discussed above.

Alternatively, the endoscope 302 may be advanced distally beyond the target tissue 330 such that the distal portion 306 of the endoscope is positioned at the desired expandable member deployment site within the trachea 344 or a bronchial tube 346. The expandable member 318 may then be advanced in a deflated configuration through a working channel (e.g., second working channel 308b) of the endoscope 302 and into the trachea 344 or bronchial tube 346. Once properly positioned within the trachea 344 or bronchial tube 346, the expandable member 318 is moved to the inflated configuration (as discussed above), and the endoscope 302 is retracted proximally to position the distal portion 306 adjacent to, or in the vicinity of, the target tissue 330. The cryogen delivery catheter 310 is then advanced distally beyond the distal portion 306 of the endoscope 302 through the first working channel 308a such that distal end 314 is adjacent to the target tissue, as discussed above.

In one embodiment, a second expandable member (not depicted) may be introduced and deployed into the bronchial tube of the non-treated lung to ensure that cryospray is properly vented through the trachea without entering the non-treated lung.

FIG. 3B depicts an alternative embodiment, in which the cryoablation system depicted in FIG. 3A includes a vent tube 326 to further assist in evacuation of the cryospray, and other undesirable fluids and materials. Although the vent tube 326 is depicted proximal to the distal portion 306 of the endoscope 302, it will be appreciated that vent tube may be positioned in a variety of locations within the trachea to facilitate active and/or passive venting of cryospray as discussed above.

In other embodiments, the cryogen delivery catheter and expandable member of the present disclosure may be combined into a single-assembly cryoablation system which allows simplified introduction into, and removal from, a body lumen either alone or through e.g., the working channel of an endoscope. It should be appreciated that all previous descriptions of dimensions, compositions and/or materials suitable for the expandable member and cryogen delivery catheter apply equally to the following embodiments.

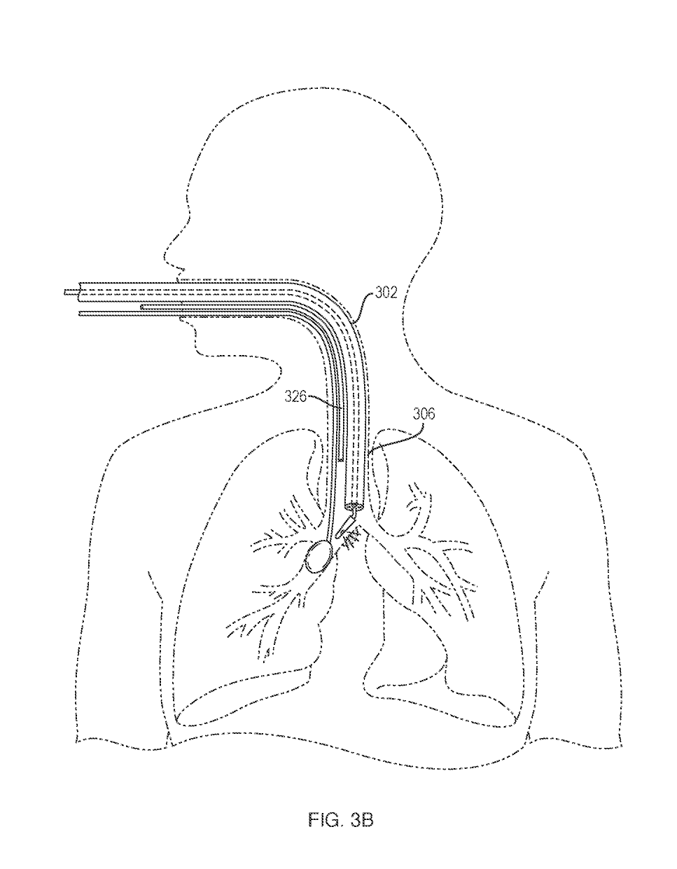

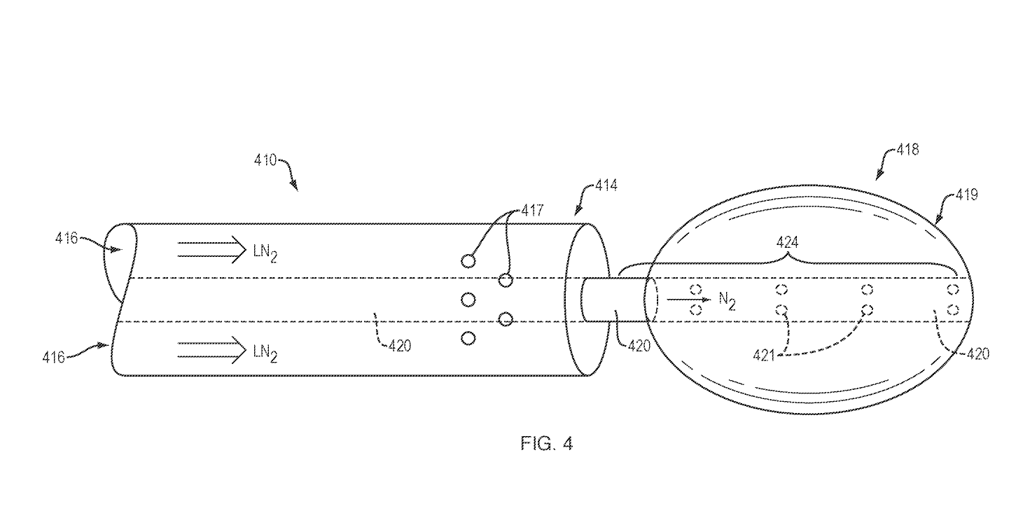

Referring to FIG. 4, in one embodiment a single-assembly cryoablation system of the present disclosure may include a cryogen delivery catheter 410 configured for delivery through a working channel of an endoscope (not depicted). The cryogen delivery catheter 410 may include a proximal end (not depicted), a distal end 414 and a lumen 416 extending therebetween. The distal end 414 may include a closed configuration with a plurality of cryogen delivery ports 417 (e.g., apertures) disposed around a portion or whole of the circumference thereof. Cryogen (e.g., liquid nitrogen) may be delivered from an external storage tank (not depicted) connected to the proximal end of the cryogen delivery catheter 410, through the lumen 416 to exit through the cryogen delivery ports 417 as a low pressure cryospray. The single-assembly cryoablation system may further include a single-lumen conduit 420 disposed within and through the lumen 416 of the cryogen delivery catheter 410. The single-lumen conduit 420 may include a proximal inlet (not depicted) fluidly connected to an external fluid source (not shown), and a distal portion 424 extending distally beyond the distal end 414. The distal portion 424 may include one or more inflation/deflation ports 421 extending through the wall of the single-lumen conduit 420 and in fluid communication with the external fluid source.

An expandable member 418 may be disposed about the distal portion 424 of the single-lumen conduit 420 such that inflation fluid may flow between the external fluid source and an interior of the expandable member 418 through the one or more inflation/deflation ports 421. For example, the expandable member 418 may move from a deflated (i.e., collapsed) configuration to an inflated (i.e., expanded) configuration by flowing an inflation fluid into the interior of the expandable member 418 through the single-lumen conduit 420. The expandable member 418 may return to the deflated configuration by flowing (e.g., returning) the inflation fluid from the interior of the expandable member 418 to the external fluid source through single-lumen conduit 420. It should be appreciated that the expandable member 418 may include an outer diameter, when in the deflated configuration, that is smaller than an outer diameter of the cryogen delivery catheter 410 to provide ease of insertion through the body lumen and/or scope working channel. The single-assembly cryoablation system may further include an outer sheath (not depicted) through which the cryogen delivery catheter 410 may be deployed and retracted, e.g., to protect the expandable member within the sheath when in the deflated configuration.

As discussed above, the inflation fluid may include a variety of physiologically inert liquids (e.g., buffered solutions such as sterile saline) or gases (e.g., air, oxygen, nitrogen, hydrogen, carbon dioxide, helium etc.) as are known in the art. It should be appreciated that due to the relatively close proximity of the cryogen delivery ports 417 to the expandable member 418, the inflation fluid may preferably exclude fluids e.g., water or saline that might freeze upon delivery of the cryospray.

In one embodiment, the single-lumen conduit 420 may include one or more centering elements (e.g., ribs etc.) configured to maintain the single-lumen conduit 420 centered within the lumen 416. In addition, or alternatively, the single-lumen conduit 420 may be supported at both the proximal end (not depicted) and distal end 414 of the cryogen delivery catheter 410 to maintain the single-lumen conduit 420 within the center of the lumen 416. For example, the closed configuration of the distal end 414 of the cryogen delivery catheter 410 may be bonded, adhered or otherwise affixed to an outer surface of the single-lumen conduit 420, which passes therethrough, to maintain the single-lumen conduit 420 within the center of the lumen 416.

In one embodiment, the inflation/deflation ports 421 may allow inflation fluid to be delivered from the external fluid source through the single-lumen conduit 420 to inflate expandable member 418, and removed under suction to deflate the expandable member 418, either manually using e.g., a syringe or automatically using an external system. The syringe (or external system) may include a pressure gauge configured to allow a medical professional to confirm that the expandable member 418 is sufficiently inflated to ensure that distal progression of cryospray is blocked, and/or sufficiently deflated for safe removal from (or repositioning within) the body lumen. For example, an automatically operated external system may include a pressure sensor configured to prevent the delivery of cryogen if the expandable member 418 is either deflated or insufficiently inflated to establish a proper seal with the tissue walls of the body lumen. Similarly, an inner or outer 419 surface of the expandable member 418 may include one or more temperature sensors and/or pressure gauges to allow the temperature and/or pressure of the expandable member to be monitored during the cryospray procedure.

In one embodiment, the fixed location of the expandable member 418 relative to the cryogen delivery ports 417 ensures that the expandable member 418 is properly positioned distally beyond the target tissue prior to the delivery of cryospray. Alternatively, the single-lumen conduit 420 may be moveable (e.g., slidable etc.) within the lumen 416 such that the distance between the cryogen delivery ports 417 and expandable member 418 may be adjusted (e.g., increased or decreased). In use, and by way of example, the single-assembly cryoablation system may be introduced into a body lumen (e.g., esophagus, colon, lungs etc.) such that the expandable member 418 is positioned in a deflated configuration distally beyond a target tissue. As discussed above, the single-assembly cryoablation system may be advanced through the body lumen alone or through the working channel of an endoscope. Once the single-assembly cryoablation system is properly positioned within the body lumen, the expandable member 418 is moved to the inflated configuration such that at least a portion of the outer surface 419 contacts all, or substantially all, of the tissues about a circumference of the body lumen wall. With the cryogen delivery ports 417 properly positioned adjacent to the target tissue, cryospray is delivered from the cryogen source through the lumen 416 of the cryogen delivery catheter such that cryospray exits one or more of the cryogen delivery ports 417 to establish cryofrost on the target tissue. The expandable member 418 may then be moved to the deflated configuration and the single-assembly cryoablation system may be either repositioned within the body lumen to treat another portion of target tissue, or removed from the patient.

As discussed above, cryospray proximal of the expandable member 418 may be vented actively or passively through a working channel of the endoscope and/or through a lumen of the cryogen delivery catheter and/or a separate vent tube. In addition, or alternatively, cryospray proximal of the expandable member 418 may be passively vented without the assistance of a vent tube or working channel by managing the body lumen and/or patient to maintain proper circulation and egress of gases.

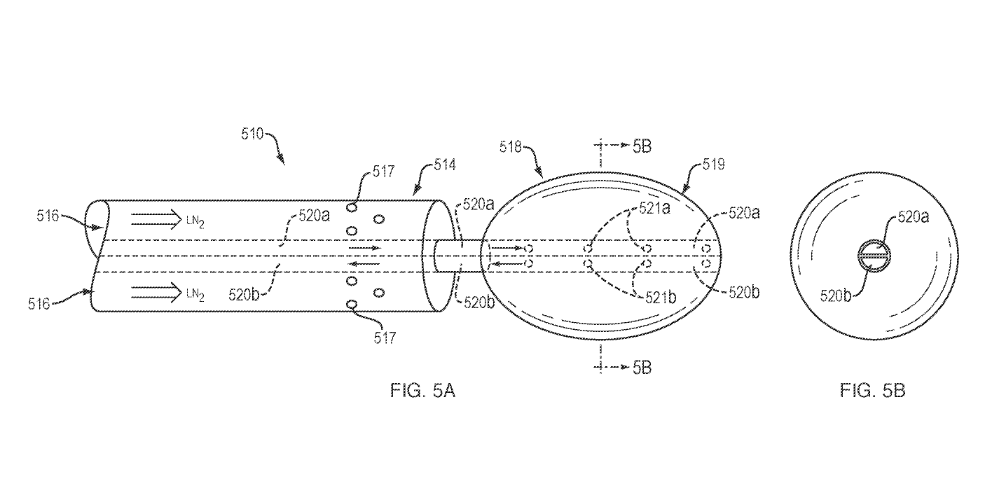

Referring to FIGS. 5A-5B, in one embodiment, a single-assembly cryoablation system may include a cryogen delivery catheter 510 comprising a dual-lumen conduit with a first lumen 520a configured to deliver inflation fluid from the external fluid source (not depicted) into the interior of the expandable member 518 through inflation ports 521a, and a second lumen 520b configured to return the inflation fluid from the interior of the expandable member 518 through deflation ports 521b to the external fluid source. For example, the expandable member 518 may move from a deflated (i.e., collapsed) configuration to an inflated (i.e., expanded) configuration by flowing an inflation fluid into the interior of the expandable member 518 through a first lumen 520a, and returned to the deflated configuration by flowing (e.g., returning) the inflation fluid from the interior of the expandable member 518 to the external fluid source through the second lumen 520b. Alternatively, inflation fluid may be continuously circulated through the first and second lumens 520a, 520b such that the expandable member 518 remains in the inflated configuration while the cryospray is being delivered to the target tissue. The inflation and deflation ports 521a, 521b may be arranged, positioned or oriented in a variety of configurations within the expandable member 518 other than the configuration depicted in FIG. 5A. By way of non-limiting example, the inflation and/or deflation ports 521a, 521b may be positioned within the proximal or distal ends of the expandable member 518. Alternatively, the inflation and deflation ports 521a, 521b may be positioned at opposite ends of the expandable member 518. It should be appreciated that the ability to continuously circulate inflation fluid through the single-assembly cryoablation system may provide a number of benefits over conventional medical devices, including, for example, the ability to continuously monitor and adjust the pressure exerted by the expandable member 518 against the body lumen wall, and the ability to minimize/prevent freezing of the inflation fluid within the expandable member and/or first and second lumens 520a, 520b.

In one embodiment, the dual-lumen conduit may include one or more centering elements (e.g., ribs etc.) configured to maintain the dual-lumen conduit centered within the lumen 516. In addition, or alternatively, the dual-lumen conduit may be supported at both the proximal end (not depicted) and distal end 514 of the cryogen delivery catheter 510 to maintain the dual-lumen conduit within the center of the lumen 516. For example, the closed configuration of the distal end 514 of the cryogen delivery catheter 510 may be bonded, adhered or otherwise affixed to an outer surface of the dual-lumen conduit, which passes therethrough, to maintain the dual-lumen conduit within the center of the lumen 516.

In one embodiment, the inflation and deflation ports 521a, 521b may allow inflation fluid to be delivered from the external fluid source through the dual-lumen conduit to inflate expandable member 518, and removed under suction to deflate the expandable member 518, either manually using e.g., a syringe or automatically using an external system. The syringe (or external system) may include a pressure gauge configured to allow a medical professional to confirm that the expandable member 518 is sufficiently inflated to ensure that distal progression of cryospray is blocked, and/or sufficiently deflated for safe removal from (or repositioning within) the body lumen. For example, an automatically operated external system may include a pressure sensor configured to prevent the delivery of cryogen if the expandable member 518 is either deflated or insufficiently inflated to establish a proper seal with the tissue walls of the body lumen. Similarly, an inner or outer 519 surface of the expandable member 518 may include one or more temperature sensors and/or pressure gauges to allow the temperature and/or pressure of the expandable member to be monitored during the cryospray procedure.

In one embodiment, the fixed location of the expandable member 518 relative to the cryogen delivery ports 517 ensures that the expandable member 518 is properly positioned distally beyond the target tissue prior to the delivery of cryospray. Alternatively, the dual-lumen conduit may be moveable (e.g., slidable etc.) within the lumen 516 such that the distance between the cryogen delivery ports 517 and expandable member 518 may be adjusted (e.g., increased or decreased). In use, and by way of example, the single-assembly cryoablation system may be introduced into a body lumen (e.g., esophagus, colon, lungs etc.) such that the expandable member 518 is positioned in a deflated configuration distally beyond a target tissue. As discussed above, the single-assembly cryoablation system may be advanced through the body lumen alone or through the working channel of an endoscope. Once the single-assembly cryoablation system is properly positioned within the body lumen, the expandable member 518 is moved to the inflated configuration such that at least a portion of the outer surface 519 contacts all, or substantially all, of the tissues about a circumference of the body lumen wall. With the cryogen delivery ports 517 properly positioned adjacent to the target tissue, cryospray is delivered from the cryogen source through the lumen 516 of the cryogen delivery catheter such that cryospray exits one or more of the cryogen delivery ports 517 to establish cryofrost on the target tissue. The expandable member 518 may then be moved to the deflated configuration and the single-assembly cryoablation system either repositioned within the body lumen to treat another portion of target tissue, or removed from the patient.

As discussed above, cryospray proximal of the expandable member 518 may be vented actively or passively through a working channel of the endoscope and/or through a lumen of the cryogen delivery catheter and/or a separate vent tube. In addition, or alternatively, cryospray proximal of the expandable member 518 may be passively vented without the assistance of a vent tube or working channel by managing the body lumen and/or patient to maintain proper circulation and egress of gases.

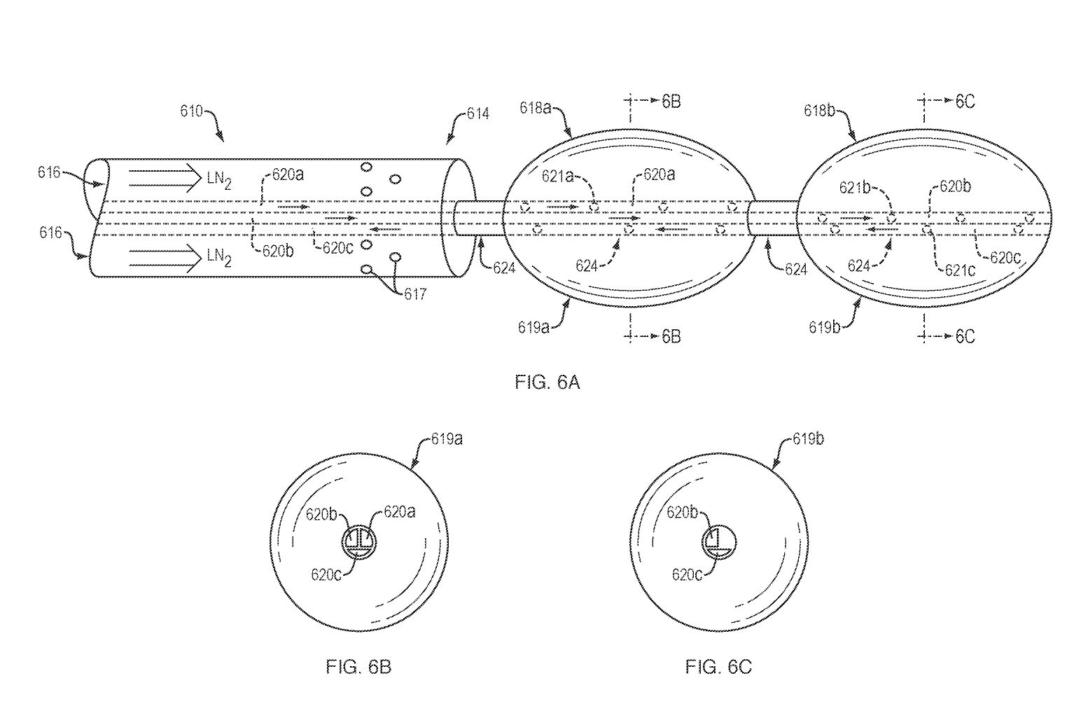

Referring to FIGS. 6A-6C, in one embodiment, a single-assembly cryoablation system of the present disclosure may include a cryogen delivery catheter 610 configured for delivery through a working channel of an endoscope (not depicted). The cryogen delivery catheter 610 may include a proximal end (not depicted), a distal end 614 and a lumen 616 extending therebetween. The distal end 614 may include a closed configuration with a plurality of cryogen delivery ports 617 (e.g., apertures) disposed around a portion or whole of the circumference thereof. Cryogen (e.g., liquid nitrogen) may be delivered from an external storage tank (not depicted) connected to the proximal end of the cryogen delivery catheter 610, through the lumen 616 to exit through the cryogen delivery ports 617 as a low pressure cryospray. The single-assembly cryoablation system may further include a multi-lumen conduit disposed within and through the lumen 616 of the cryogen delivery catheter 610. The multi-lumen conduit may include a proximal inlet (not depicted) fluidly connected to an external fluid source (not depicted), and a distal portion 624 extending distally beyond the distal end 614. First and second expandable members 618a, 618b may be disposed about the distal portion 624 of the multi-lumen conduit. The multi-lumen conduit may include a first lumen 620a configured to deliver inflation fluid from the external fluid source (not depicted) into the interior of the first expandable member 618a through inflation ports 621a, a second lumen 620b configured to deliver inflation fluid from the external fluid source into the interior of the second expandable member 618b through inflation ports 621b, and a third lumen 620c configured to return the inflation fluid from interior of the first and second expandable members 618a, 618b to the external fluid source through deflation ports 621c. For example, the first and second expandable members 618a, 618b may move from a deflated (i.e., collapsed) configuration to an inflated (i.e., expanded) configuration by flowing an inflation fluid through the respective first and second lumens 620a, 620b and inflation ports 621a, 621b.

It should be appreciated that the separate first and second lumens 620a, 620b may allow the first and second expandable members 618a, 618b to be inflated simultaneously or independent of each other. The first and second expandable members 618a, 618b may return to the deflated configuration by flowing (e.g., returning) the inflation fluid from the respective lumens of each expandable member through the deflation ports 621c and third lumen 620c to the external fluid source. Alternatively, inflation fluid may be continuously circulated through the first, second and third lumens 620a-c, such that the first and second expandable members 618a, 618b remain in the inflated configuration while the cryospray is being delivered to the target tissue. In another embodiment, each of the first and second expandable members 618a, 618b may include dedicated inflow and outflow lumens to provide independent inflation and deflation. It should be appreciated that the first and second expandable members 618a, 618b may include an outer diameter, when in the deflated configuration, that is smaller than an outer diameter of the cryogen delivery catheter 610 to provide ease of insertion through the body lumen and/or scope working channel. It should also be appreciated that the ability to continuously circulate inflation fluid through the single-assembly cryoablation system may provide a number of benefits over conventional medical devices, including, for example, the ability to continuously monitor and adjust the pressure exerted by each of the first and second expandable members 618a, 618b against the body lumen wall, and the ability to minimize/prevent freezing of the inflation fluid within the expandable member and/or first, second and thirds lumens 620a-c. The first, second and third lumens 620a-c may be arranged, positioned or oriented in a variety of configurations and shapes beyond the configuration and shape depicted in FIGS. 6A-6B. Similarly, the inflation ports 621a, 621b and deflation ports 621c may be arranged, positioned or oriented in a variety configurations within respective first and second expandable members 618a, 618b other than the configuration depicted in FIG. 6A. By way of non-limiting example, the inflation ports 621a, 621b and deflation ports 621c may be positioned within the proximal or distal ends of the first and second expandable members 618a, 618b. Alternatively, the inflation ports 621a, 621b and deflation ports 621c may be positioned at opposite ends of the expandable members 618a, 618b.

In one embodiment, the fixed location of the first and second expandable members 618a, 618b relative to the cryogen delivery ports 617 ensures that the first and second expandable members 618a, 618b are properly positioned distally beyond the target tissue prior to the delivery of cryospray. In use, and by way of example, the single-assembly cryoablation system may be introduced into a body lumen (e.g., esophagus, colon, lungs etc.) such that the first and second expandable members 618a, 618b are positioned in a deflated configuration distally beyond a target tissue. As discussed above, the single-assembly cryoablation system may be advanced through the body lumen alone or through the working channel of an endoscope. Once the single-assembly cryoablation system is properly positioned within the body lumen, the first and second expandable members 618a, 618b are moved to the inflated configuration, either simultaneously or individually, such that at least a portion of the outer surface 619a, 619b the first and second expandable members 618a, 618b contact all, or substantially all, of the tissues about a circumference of the body lumen wall. With the cryogen delivery ports 617 properly positioned adjacent to the target tissue, cryospray is delivered from the cryogen source through the lumen 616 of the cryogen delivery catheter such that cryospray exits one or more of the cryogen delivery ports 617 to establish cryofrost on the target tissue. The first and second expandable members 618a, 618b may then be moved to the deflated configuration and the single-assembly cryoablation system either repositioned within the body lumen to treat another portion of target tissue, or removed from the patient.

In one embodiment, the multi-lumen conduit may include one or more centering elements (e.g., ribs etc.) configured to maintain the multi-lumen conduit centered within the lumen 616. In addition, or alternatively, the multi-lumen conduit may be supported at both the proximal end (not depicted) and distal end 614 of the cryogen delivery catheter 610 to maintain the multi-lumen conduit within the center of the lumen 616. For example, the closed configuration of the distal end 614 of the cryogen delivery catheter 610 may be bonded, adhered or otherwise affixed to an outer surface of the multi-lumen conduit, which passes therethrough, to maintain the multi-lumen conduit within the center of the lumen 616.

In one embodiment, the inflation ports 621a, 621b may allow inflation fluid to be delivered from the external fluid source through the first and second lumens 620a, 620b to inflate the first and second expandable members 618a, 618b, and removed under suction to deflate the first and second expandable members 618a, 618b, either manually using e.g., a syringe or automatically using an external system. The syringe (or external system) may include a pressure gauge configured to allow a medical professional to confirm that the first and second expandable members 618a, 618b are sufficiently inflated to ensure that distal progression of cryospray is blocked, and/or sufficiently deflated for safe removal from (or repositioning within) the body lumen.

For example, with respect to any of the embodiments, an automatically operated external system may include a pressure sensor configured to prevent the delivery of cryogen if either of the first or second expandable members 618a, 618b are either deflated or insufficiently inflated to establish a proper seal with the tissue walls of the body lumen. Similarly, an inner or outer 619a, 619b surface of the first and second expandable members 618a, 618b may include one or more temperature sensors and/or pressure gauges to allow the temperature of the expandable member to be monitored during the cryospray procedure.

As discussed above, cryospray proximal of the first expandable member 618a may be vented through a working channel of the endoscope and/or through a vent tube in the cryogen delivery catheter or a separate vent tube. In addition, or alternatively, cryospray proximal of the first expandable member 618a may be passively vented without the assistance of a vent tube or working channel by managing the body lumen and/or patient to maintain proper circulation and egress of gases.

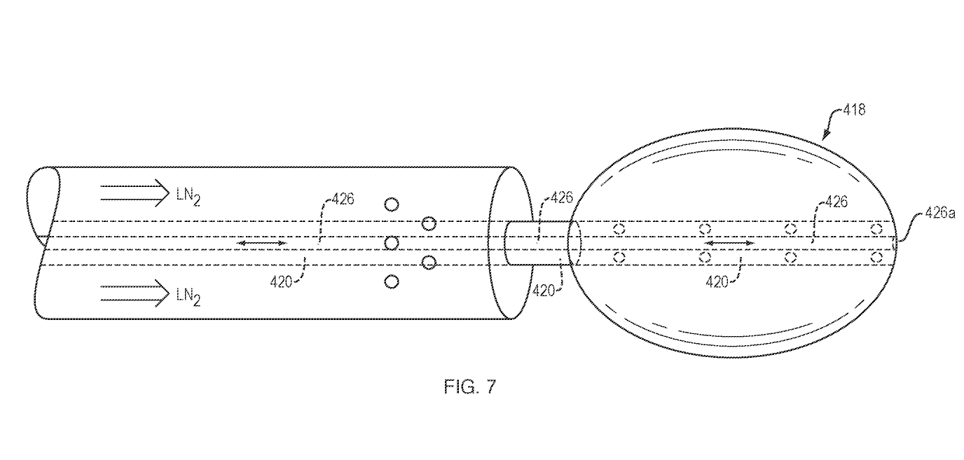

Referring to FIG. 7, in one embodiment, a single-assembly cryoablation system, such as that of FIG. 4, may further include a vent tube 426 which passes through the length of the single-lumen conduit 420, and passes through and extends distally beyond expandable member 418. In this configuration, the distal opening 426a of vent tube 426 may actively and/or passively vent cryospray that has advanced distally beyond the expandable member 418. The expandable member 418 forms a tight seal around the outer surface of the vent tube 426 such that the respective lumens of the expandable member and vent tube remain separated and uncompromised.

It should be appreciated that such a vent tube is not limited to the single-assembly cryoablation system of FIG. 4, but may be included in either of the single-assembly cryoablation systems depicted in FIGS. 5 and 6, or other embodiments. For example, the single-assembly cryoablation system of FIG. 6 may include a vent tube which passes through the length of the multi-lumen conduit, and passes through the first and second expandable members 618a, 618b and extends distally beyond the second expandable member 618b. In addition, or alternatively, a second vent tube may pass through the length of the multi-lumen conduit, and extend through and distally beyond the first expandable member 618a with an inlet between first and second expandable members 618a, 618b. As discussed above, such vent tubes may actively and/or passively vent cryospray that has advanced distally beyond either of the first and/or second expandable members 618a, 618b. It should also be appreciated that the vent tube, inflation lumens, deflation lumens and/or cryogen delivery catheters disclosed herein may be independently moveable relative to each other such that the distance between the cryospray and expandable member(s), and/or the distance between the vent tube and expandable member may be adjusted prior to or during the medical procedure.

It should be appreciated that any of the embodiments described herein that include a vent tube 126, 226, 326 which extends proximal to an expandable member (FIGS. 1B, 2B, 2C and 3B), or which passes through and extends distally beyond an expandable member (FIG. 1C), may further benefit from passive or active venting of the treatment area (i.e., proximal to the expandable member) through a working channel of the endoscope and/or a working channel of the cryogen delivery catheter. It should further be appreciated that passive venting may be further facilitated, independent of such vent tubes and/or working channel(s), by managing the body lumen to maintain proper circulation and egress of gases, as discussed above.

All of the devices and/or methods disclosed and claimed herein can be made and executed without undue experimentation in light of the present disclosure. While the devices and methods of this disclosure have been described in terms of preferred embodiments, it may be apparent to those of skill in the art that variations can be applied to the devices and/or methods and in the steps or in the sequence of steps of the method described herein without departing from the concept, spirit and scope of the disclosure. All such similar substitutes and modifications apparent to those skilled in the art are deemed to be within the spirit, scope and concept of the disclosure as defined by the appended claims.

What is claimed is:

1. A cryoablation system, comprising:
an endoscope comprising:
a proximal portion,
a distal portion, and
at least one working channel extending therebetween;
a cryogen delivery catheter disposable within a working channel of the endo scope, the cryogen delivery catheter comprising:
a proximal end,
a distal end configured for the output of cryogen spray, and
a lumen extending therebetween;
an expandable member moveable between a deflated configuration and an inflated configuration and including a conduit comprising a proximal inlet and a distal outlet, wherein the distal outlet is fluidly connected to an interior of the expandable member; and
a vent tube configured for passive or active venting of a gas therethrough that extends distally beyond the distal portion of the endoscope and through the expandable member.

2. The cryoablation system of claim 1, wherein the expandable member is configured to extend distally beyond the distal portion of the endoscope.

3. The cryoablation system of claim 1, wherein the expandable member moves from the deflated configuration to the inflated configuration by flowing a fluid into the interior of the expandable member.

4. The cryoablation system of claim 1, wherein the expandable member comprises a compliant material selected from the group consisting of silicone rubbers, polyurethanes, butyl rubbers, latexes, styrene-isobutylene-styrene block copolymers and EPDM.

5. The cryoablation system of claim 1, wherein the expandable member conforms to the shape of a body lumen when in the inflated configuration and prevents or substantially inhibits gas progression distally beyond the expandable member.

6. The cryoablation system of claim 1, wherein the vent tube is independent of the working channel of the endoscope.

7. A cryoablation system, comprising:
a cryogen delivery catheter comprising:
a proximal end,
a distal end including an outlet for cryogen, and
a lumen extending therebetween;
a single-lumen conduit disposed within the lumen, the single-lumen conduit comprising:
a distal portion extending distally beyond the cryogen outlet at the distal end of the cryogen delivery catheter, wherein the distal portion includes at least one port in fluid communication with the single-lumen conduit; and
an expandable member disposed about the distal portion of the single-lumen conduit and defining an interior, wherein the expandable member is moveable between an unexpanded configuration and an expanded configuration.

8. The cryoablation system of claim 7, wherein the expandable member moves from the unexpanded configuration to the expanded configuration by flowing a fluid through the single-lumen conduit, and the at least one port, into the interior of the expandable member.

9. The cryoablation system of claim 7, wherein the expandable member moves from the expanded configuration to the unexpanded configuration by flowing a fluid from the interior of the expandable member through the at least one port and the single-lumen conduit.

10. The cryoablation system of claim 7, further comprising a vent tube disposed within the lumen of the cryogen delivery catheter, wherein a distal portion of the vent tube passes through and extends distally beyond the expandable member.

11. The cryoablation system of claim 7, wherein the expandable member is a balloon and comprises a non-compliant or semi-compliant material.

12. The cryoablation system of claim 11, wherein the non-compliant or semi-compliant material comprises a polymer selected from the group consisting of PEBAX, PET, PEN, PBT, PEEK, Hytrel, polyurethane and nylon.

13. A cryoablation system, comprising:
a cryogen delivery catheter comprising:
a proximal end,
a distal end configured for the output of cryogen, and
a lumen extending therebetween;
a multi-lumen conduit disposed within the lumen of the cryogen delivery catheter, the multi-lumen conduit comprising:
a distal portion extending distally beyond the distal end of the cryogen delivery catheter, wherein the distal portion includes at least one port in fluid communication with a first lumen of the multi-lumen conduit and at least one port in fluid communication with a second lumen of the multi-lumen conduit; and
a first expandable member disposed about the distal portion of the multi-lumen conduit and defining an interior, wherein the first expandable member is moveable between a deflated configuration and an inflated configuration.

14. The cryoablation system of claim 13, wherein the first expandable member moves from the deflated configuration to the inflated configuration by flowing a fluid through the first lumen of the multi-lumen conduit, and the at least one first lumen port, into the interior of the first expandable member.

15. The cryoablation system of claim 13, wherein the first expandable member moves from the inflated configuration to deflated configuration by flowing a fluid from the interior of the first expandable member through the at least one second lumen port and the second lumen of the multi-lumen conduit.

16. The cryoablation system of claim 13, further comprising a vent tube disposed within the lumen of the cryogen delivery catheter, wherein a distal portion of the vent tube passes through and extends distally beyond the first expandable member.

17. The cryoablation system of claim 13, wherein the first expandable member comprises a non-compliant or semi-compliant material.

18. The cryoablation system of claim 17, wherein the non-compliant or semi-compliant material comprises a polymer selected from the group consisting of PEBAX, PET, PEN, PBT, PEEK, Hytrel, polyurethane and nylon.

19. The cryoablation system of claim 13, further comprising:
a second expandable member disposed about the distal portion of the multi-lumen conduit and defining an interior, wherein the second expandable member is moveable between a deflated configuration and an inflated configuration; and
a third lumen of the multi-lumen conduit in fluid communication with the interior of the first expandable member and the second expandable member;
wherein the second lumen is in fluid communication with the interior of the second expandable member; and
wherein the first lumen is in fluid communication with the interior of the first expandable member.

20. The cryoablation system of claim 19,
wherein the first expandable member transitions from the deflated configuration to the inflated configuration by flowing a fluid through the first lumen to the interior of the first expandable member;
wherein the second expandable member transitions from the deflated configuration to the inflated configuration by flowing a fluid through the second lumen to the interior of the second expandable member; and
wherein the first and second expandable members transition from the inflated configuration to deflated configuration by flowing a fluid from interior of the first and second expandable members through the third lumen.
